(19) United States
(12) Plant Patent Application Publication
(10) Pub. No.: US 2003/0213039 P1
(43) Pub. Date: Nov. 13, 2003

(54) Plant patent for delosperma plant named White Nugget

(76) Inventors: Kelly Grummons, Lakewood, CO (US); Cheryl Vestal, Anada, CO (US)

Correspondence Address: LEE G. MEYERS, MEYERS AND ASSOCIATES, P.C., 17462 E. POWERS DRIVE, CENTENNIAL, CO, 80015-3046, US

(21) Appl. No.: 10/143,187

(22) Filed: May 9, 2002

(51) Int. Cl. A01H 5/00

(52) U.S. Cl. PLT/263

(57) ABSTRACT

The present invention relates to a new and distinct variety of Delosperma named White Nugget, the novel characteristics of which reside particularly in its low and compact growing pattern, compact glossy pale foliage, unique white flower, exceptional cold-hardiness, repeated blooming pattern throughout the spring and ability to grow well in a wide-variety of soils and regions.

### BACKGROUND OF THE INVENTION

The present invention relates to a new and distinct cultivar of Ice Plant, botanically known as Delosperma, and referred to by the cultivar name White Nugget.

The new cultivar was selected from seedlings which resulted from a cross of Delosperma “Gold Nugget” and perhaps other unknown yellow-flowered parents of Delosperma, and was selected because of its pure white color and exceptional cold-hardiness.

The new cultivar was first discovered by Kelly Grummons in 1996 at Paulino Gardens of Denver, Colo. It was then asexually reproduced in 1997 by Cheryl J. Vestal at Paulino Gardens. Asexual reproduction of the new cultivar by terminal cuttings has shown that the unique features of this new Delosperma are stable, and are reproduced true to type in successive propagations.

The unique characteristics of White Nugget have been repeatedly observed and are determined to be basic characteristics of this cultivar, which in combination, distinguish this delosperma from other Delosperma species, such as the cultivar Delosperma “Gold Nugget.”

An interesting characteristic of the new variety is its low, compact spreading habit. White Nugget, like its parent Gold Nugget, is consistently lower and more dense-growing than the other commonly grown Delospermas commercially available, such as D. cooperi, D. nubigenum or D. floribundum.

Another interesting characteristic of the new variety is the pale foliage. White Nugget has slightly less pigment in the foliage than other ice plants, like many albino sports.

Another interesting characteristic of the new variety is the pure white flowers. Flowers of White Nugget are pure white and do not contain any other pigment. All other commonly cultivated Delosperma varieties are pink or an impure white. In addition, other varieties of Delosperma that display white flowers do not display cold-hardiness and are also somewhat shrubby in appearance. These other varieties are easily distinguishable from the new variety described herein.

Another interesting characteristic of the new variety is its exceptional cold-hardiness. White Nugget exhibits unusual cold tolerance when compared to other Delosperma species, exhibiting hardiness to at least 20 degrees below zero.

Another interesting characteristic of the new variety is its early flowering season. White Nugget produces blooms in early spring and they last until well into the summer. They bloom much earlier and also bloom much longer than any other variety of Delosperma, except for Gold Nugget.

### BRIEF SUMMARY OF THE INVENTION

The principal novel characteristic of the new variety of Delosperma is its pure white flower. The new variety of Delosperma is further characterized by its low, compact spreading habit, pale foliage, exceptional cold-hardiness and early flowering season. All of these characteristics work to provide a variety of Delosperma that is visually pleasing with its abundance of white flowers and easy to grow due to its ability to spread easily and withstand cold temperatures.

### BRIEF DESCRIPTION OF THE PICTURES

The accompanying photographic drawings show typical flower and leaf characteristics of White Nugget, with the colors being as nearly true as possible with illustrations of this type. The distinctive characteristics of the new variety are exemplified in the accompanying illustrations, wherein:

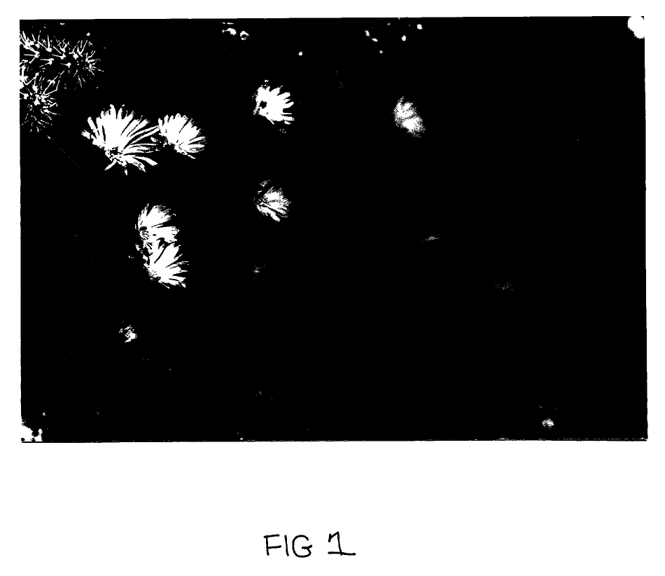

FIG. 1 shows White Nugget in bloom. Some of the blooms are open and others are closed or partially closed.

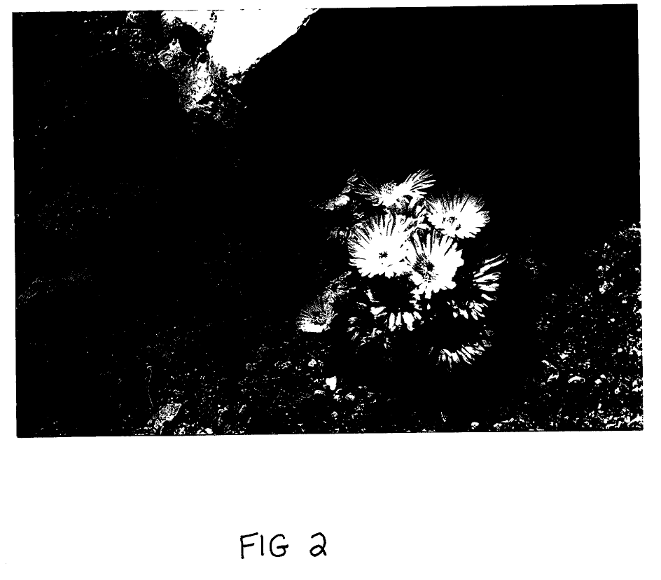

FIG. 2 is a close up of White Nugget in bloom.

### DETAILED BOTANICAL DESCRIPTION

The following is a description in some detail of the new variety of Delosperma. All color references made in the description refer to The Royal Horticultural Society Colour Chart except where general descriptive terms are used. The color values were determined on plant material grown in Denver, Colo. on Apr. 23, 2002.

Botanical classification: Delosperma cultivar named “White Nugget”.

Parentage: Clearly Delosperma “Gold Nugget” and possibly another unidentified species of delosperma.

Propagation:

Type.—By cuttings.

Time to initiate roots.—12 to 16 days.

Rooting habit.—Fibrous and branching.

Plant description:

General appearance.—Compact spreading mound, (10 to 25 cm) with dense foliage and stemless flower. White Nugget has a basal branching habit and slow to moderate growth rate.

Foliage description.—Arrangement: single, alternate. Leaf shape: Oblanceolate, terete, with attenuate leaf base and rounded or slightly pointed apex. Leaf margin: Smooth. Leaf dimension: Range from 11 to 34 m in length, and 2.5 to 7 mm wide. Leaf color: Upper surface: Yellow-Green group 147A. Lower surface: Yellow-Green group 147B. Foliage can take on reddish overtones during the winter cold months if kept outdoors but stays a strong green if kept under glass.

Flowering description:

Inflorescence.—Superficially resembles a daisy with numerous ray-like petals.

Flowering habit.—Flowers freely.

Flowering season.—Blooms heavily from March to May with scattered blooms later in the season.

Buds.—Pointed, glabrous, 3 to 12 mm in length, 5 mm across.

Flower color.—Upper surface opens a uniform white and undersurface is also white.

Petals.—Number: 42 to 57. Shape: Lanceolate. Margin: Entire. Tip: Rounded.

Texture.—Smooth.

Appearance.—Satiny.

Length.—7 to 13 mm.

Width.—1 to 2 mm.

Overall flower diameter.—18 to 28 mm.

Reproductive organs: A distinct, five sectioned ovary that develops into an almost woody, multi-seeded capsule with one style protruding slightly from the corolla. Style branches are white.

1. A new and distinctive variety of Delosperma, substantially as herein shown and described, characterized particularly as to novelty by the unique combination of its ability to exhibit low and compact spreading, exhibit pale foliage, demonstrate compact glossy foliage, exhibit exceptional cold-hardiness, exhibit blooms repeatedly all spring, ability to grow well in a variety of soils, ability to grow well in a variety of regions and its novel white flowers.
